(19) United States
(12) Patent Application Publication
(10) Pub. No.: US 2022/0313204 A1
(43) Pub. Date: Oct. 6, 2022

(54) ULTRASONIC CT DEVICE, METHOD FOR CONTROLLING THE SAME, AND PROGRAM FOR CONTROLLING ULTRASONIC CT DEVICE

(71) Applicant: FUJIFILM Healthcare Corporation, Kashiwa-shi (JP)

(72) Inventors: Takahide TERADA, Chiba (JP); Yushi TSUBOTA, Chiba (JP); Atsuro SUZUKI, Chiba (JP)

(21) Appl. No.: 17/590,882

(22) Filed: Feb. 2, 2022

(30) Foreign Application Priority Data
Mar. 30, 2021 (JP) 2021-058547

(51) Int. Cl. A61B 8/08; G01S 15/89; G01S 7/52; A61B 8/00

(52) U.S. Cl. CPC A61B 8/0825; G01S 15/8934; G01S 15/8993; G01S 7/52053; A61B 8/543

(57) ABSTRACT

Provided is an ultrasonic CT device capable of shortening a transmission interval of ultrasonic waves while suppressing influence of a reception signal of reverberation waves of the ultrasonic waves on an original reception signal. when transmission and reception are repeated, a timing of current transmission is controlled such that a timing at which reverberation waves, which are ultrasonic waves transmitted in previous transmission and are reflected by a transducer array at least once, reach transducers used for current reception deviates from a timing at which ultrasonic waves transmitted in current transmission reach the transducers used for the current reception.

### BACKGROUND OF THE INVENTION

### 1. Field of the Invention

The present invention relates to an ultrasonic CT device, in particular, to a method for controlling the same.

### 2. Description of the Related Art

WO 2017/098641 (Patent Literature 1) and the like discloses an ultrasonic computed tomography (CT) device that is an ultrasonic imaging device that generates a tomographic image of an object. The object is placed in an ultrasonic wave propagation medium (such as water) that is a medium that propagates ultrasonic waves, and transducers are arranged to surround the object (for example, in a ring shape). The ultrasonic waves are transmitted from the transducers at various positions toward an inside of the object, ultrasonic waves scattered on a surface or the inside of the object or ultrasonic waves passing through the inside of the object are received by the transducers at various positions. Based on a reception signal, distribution of a physical value (such as sound speed) that reflects a shape and acoustic characteristics of the object is calculated, and a physical value distribution image (sound speed image) or the like of the object is generated.

Olivier Roy, Steven Schmidt, Cuiping Li, Veerendra Allada, Erik West, David Kunz, and Neb Duric, “Breast imaging using ultrasound tomography: From clinical requirements to system design”, 2013 IEEE International Ultrasonics Symposium (IUS), pp. 1174-1177, 2013 (Non-Patent Literature 1) discloses that ultrasonic waves are transmitted at intervals of 800 μs, and an ultrasonic signal passing through an object (passing waves) is received to generate a sound speed image.

In an ultrasonic CT device, a transducer array in which transducers are arranged to surround a measurement object is used. For example, a shape of the transducer array is a ring shape. Ultrasonic waves transmitted from a transducer pass through an object, reach the transducer array, and are received. A part of the ultrasonic waves are reflected by the transducer array they reached, and the reflected ultrasonic waves pass through the object again and reach another transducer. A part of the reflected ultrasonic waves are reflected again by the transducer array. Ultrasonic waves reflected at least once by the transducer array are referred to as “reverberation waves” in the present specification. That is, not only the ultrasonic waves transmitted from a transducer, passing through the object once, and reaching the transducer array, but also the reverberation waves reflected at least once by the transducer array reach and are received by the transducer array. The reverberation waves are noise when a shape and acoustic characteristics of the measurement object are imaged.

Since the ultrasonic CT device uses a ring-shaped transducer array, the ultrasonic waves are reflected multiple times and reciprocate in a complicated manner, the reverberation waves are likely to be generated structurally, and a shape of a wavefront of the ultrasonic waves reflected by a curved surface is also complicated. In addition, in the ultrasonic CT device, since the ultrasonic waves are transmitted plural times from an entire circumference of the measurement object while changing positions of the transducers from which the ultrasonic waves are transmitted, propagation directions of the reverberation waves also change each time the positions of the transducers from which the ultrasonic waves are transmitted are changed. Furthermore, to reduce the number of channels in a processing circuit, the ultrasonic CT device does not perform signal processing for reception signals from all the transducers of the ring-shaped transducer array at once, but divides the transducer array into a plurality of groups, repeats transmission from the same transducers a plurality of times, and sequentially obtains the reception signals for each group to perform signal processing. Therefore, it is necessary to repeat the transmission from the same transducers a plurality of times, and the number of transmissions is further increased. Therefore, the reverberation waves are generated every time a large number of transmissions are performed, and there are plural combinations of positions of a group of transmission transducers and a group of reception transducers, and the reverberation waves are generated in a complicated manner.

In the related art, to prevent a reception signal of reverberation waves of previously transmitted ultrasonic waves from overlapping with a reception signal of currently transmitted ultrasonic waves, next transmission is performed after waiting for the previous reverberation waves to be sufficiently attenuated. Therefore, for example, a transmission interval of about 800 μs is necessary, and it is difficult to shorten imaging time of ultrasonic CT that repeats the transmission and reception plural times.

### SUMMARY OF THE INVENTION

An object of the invention is to provide an ultrasonic CT device capable of shortening a transmission interval of ultrasonic waves while suppressing influence of a reception signal of reverberation waves of the ultrasonic waves on an original reception signal.

In order to solve the above problem, an ultrasonic CT device according to the invention includes: a transducer array in which a plurality of transducers are arranged to surround a region where a measurement object is placed; a transmitter configured to output a transmission signal to the transducers to transmit ultrasonic waves; a receiver configured to receive and process reception signals obtained by a predetermined number of transducers of the transducer array receiving and outputting the ultrasonic waves from the region that received the ultrasonic waves; and a control unit configured to control repetition of transmission and reception of the ultrasonic waves in which combinations of the transducers from which the ultrasonic waves are transmitted and the transducers that receive the reception signals are sequentially set in the transmitter and the receiver according to a predetermined series of combinations. When the transmission and reception are repeated, the control unit controls a timing of current transmission such that a timing at which reverberation waves, which are ultrasonic waves transmitted in previous transmission and are reflected by the transducer array at least once, reach transducers used for current reception deviates from a timing at which ultrasonic waves transmitted in the current transmission reach the transducers used for the current reception.

According to the invention, since a transmission timing is set for each transmission so that the timing at which the ultrasonic waves reach the reception transducers avoids the timing at which the previous reverberation waves reach the reception transducers, the transmission can be performed without waiting until the reverberation waves are sufficiently attenuated, and the imaging time can be shortened.

### BRIEF DESCRIPTION OF THE DRAWINGS

FIG. 1 is a block diagram showing an overall configuration of an ultrasonic CT device according to a first embodiment of the invention.

FIG. 2 is a functional block diagram of the ultrasonic CT device according to the first embodiment.

FIGS. 3A to 3E are schematic diagrams showing simulation results of wavefronts of ultrasonic waves and reverberation waves in a ring-shaped transducer array of the ultrasonic CT device according to the first embodiment.

FIG. 4 is a graph showing timings at which the ultrasonic waves and the reverberation waves reach first to seventh reception modules of the transducer array of the ultrasonic CT device according to the first embodiment.

FIG. 5 is a graph showing signal intensities of passing waves (the ultrasonic waves) and the reverberation waves (the number of times of reflection: 1 to 4 times) of the ultrasonic CT device according to the first embodiment.

FIG. 6 is a graph showing reception timings avoiding reaching timings of the reverberation waves to the first to seventh reception modules of the ultrasonic CT device and resultant transmission timings according to the first embodiment.

FIG. 7 is a schematic diagram showing transmission timings and reception timings of a series of transmission and reception in which reception is sequentially performed by the first to seventh reception modules of the ultrasonic CT device according to the first embodiment.

FIG. 8 is a flowchart showing operations of the ultrasonic CT device according to the first embodiment.

FIG. 9 is a diagram showing a UI screen of an ultrasonic CT device according to a second embodiment.

FIG. 10 is a flowchart showing operations of an ultrasonic CT device according to a third embodiment.

FIG. 11 is a flowchart showing operations of an ultrasonic CT device according to a fourth embodiment.

### DESCRIPTION OF THE PREFERRED EMBODIMENTS

An ultrasonic CT device according to one embodiment of the invention will be described.

### First Embodiment

In the invention, a propagation path of reverberation waves is estimated focusing on the fact that arrangements of transducers of a ring-shaped transducer array are known and that reverberation waves are almost specularly reflected since a surface of the ring-shaped transducer array is smooth. Accordingly, a timing at which reverberation waves of previously transmitted ultrasonic waves reach the transducers is estimated, and a transmission timing of ultrasonic waves to be currently transmitted is set such that a timing (reception period) at which the currently transmitted ultrasonic waves reach transducers for current reception does not overlap with the estimated timing. Accordingly, the ultrasonic waves can be transmitted without waiting until previous reverberation waves are sufficiently attenuated. The reverberation waves here mean sound waves obtained by reflecting previously transmitted ultrasonic waves (N−1th time and earlier) before current transmission (N-th time) by the transducer array at least once.

Specifically, when an image is taken by repeating a predetermined series of transmission and reception, the ultrasonic CT device of the present embodiment controls a timing of the current transmission such that a timing at which the previously transmitted reverberation waves reflected by the transducer array at least once reach the transducers for the current reception deviates from a timing at which the currently transmitted ultrasonic waves reach the transducers for the current reception.

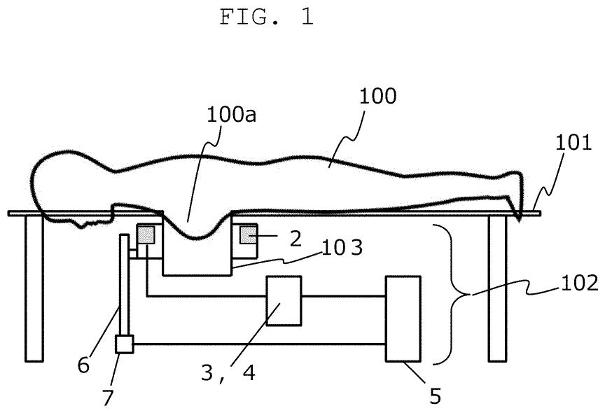

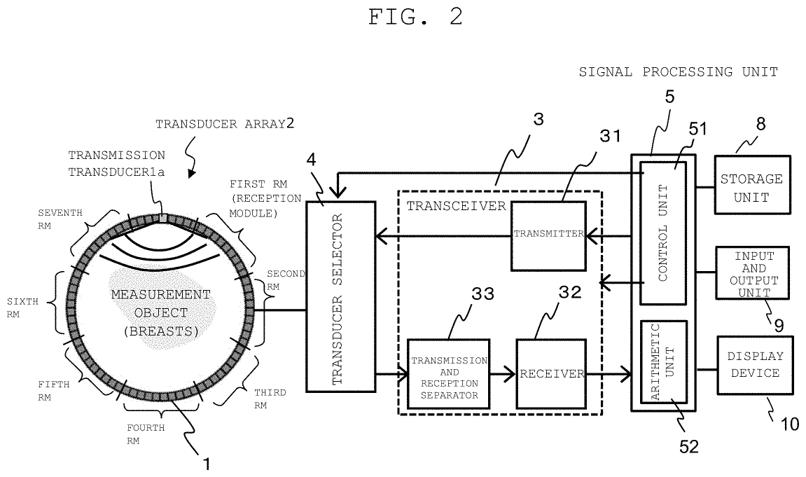

Hereinafter, the ultrasonic CT device of the present embodiment will be described. As shown in FIGS. 1 and 2, the ultrasonic CT device of the present embodiment is an ultrasonic CT device for breasts, and includes a bed 101 on which a subject 100 is placed facing downward, and a measurement unit 102 disposed below an opening provided in the bed 101.

The measurement unit 102 includes a container 103 filled with water, a transducer array 2 in which transducers 1 are arranged in a ring shape, a plurality of transceivers 3, a transducer selector 4, a signal processing unit 5, a storage unit 8, a mechanism unit 6 that vertically moves the transducer array 2, and a drive unit 7 of the mechanism unit 6. Although only one transceiver 3 is shown in FIG. 2, the plurality of transceivers (for example, 256 channels) are provided. The number of the transducers 1 in the transducer array 2 is larger than the number of the transceivers 3, and is, for example, 2048 channels.

The container 103 is disposed below the bed 101 such that an opening matches the opening of the bed 101. Accordingly, breasts 100a of the subject 100 are inserted into the container 103 through the opening of the bed 101. The transducer array 2 is disposed inside or outside the container 103.

Each of the transceivers 3 includes a transmitter 31, a receiver 32, and a transmission and reception separator 33. The signal processing unit 5 includes a control unit 51 and an arithmetic unit 52. The signal processing unit 5 is connected to an input and output unit 9 that receives imaging conditions and the like from a user, the storage unit 8, and a display device 10 that displays a generated CT image and the like.

At the time of transmission, the transducer selector 4 selectively connects one or more transducers 1a from which ultrasonic waves in the current transmission are to be transmitted to the transmitter 31. At the time of reception, the transducer selector 4 selectively connects one or more transducers 1 to which the receiver 32 are currently to receive a reception signal to the receiver.

The storage unit 8 stores in advance, for each of multiple times of transmission and reception required to obtain one ultrasonic CT image, a combination of positions of the transducers 1a from which the ultrasonic waves are transmitted and positions of a plurality of transducers 1 that receive the reception signal, and a transmission interval.

Each time transmission and reception are performed, the control unit 51 reads out a combination of the transducers 1 used for transmission and the transducers 1 that receive the reception signal read from the storage unit 8, and instructs the transducer selector 4.

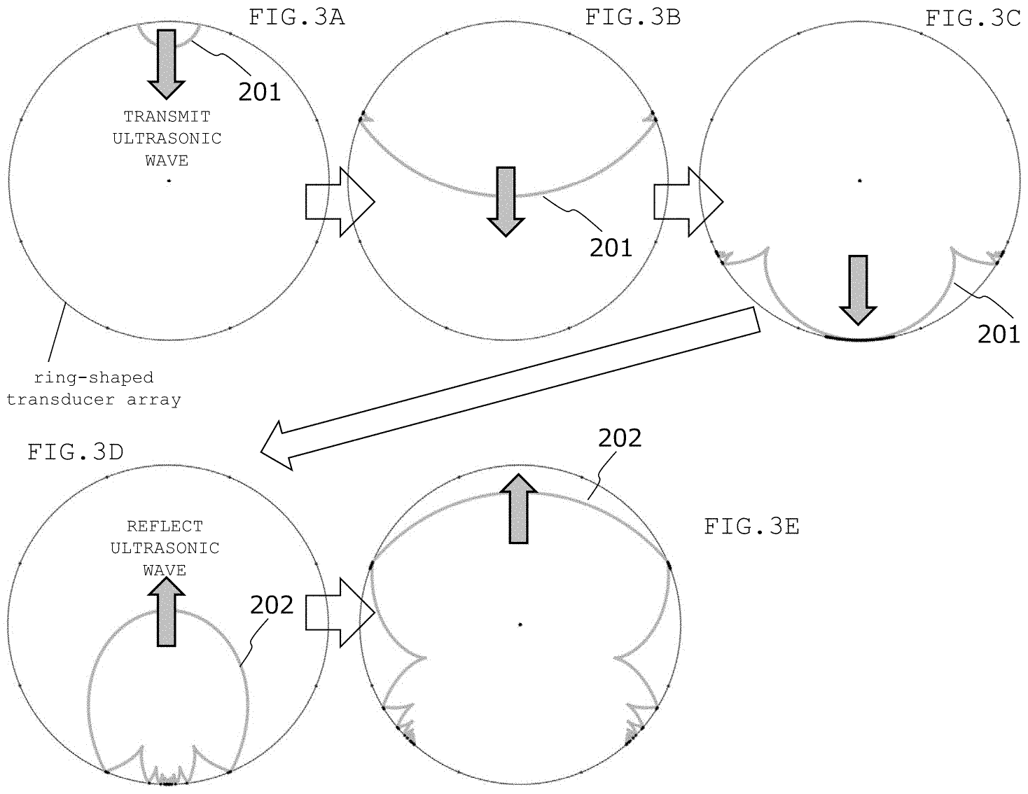

The transmitter 31 receives an electric signal transmitted from the control unit 51, amplifies the signal to generate a transmission signal, and outputs the transmission signal to the transducers 1 used for the transmission via the transducer selector 4. The transducers that receive the transmission signal converts the transmission signal into ultrasonic waves 201 and transmits the ultrasonic waves 201 to a space where the breasts 100a are placed (FIG. 3A).

A part of the transmitted ultrasonic waves 201 are scattered and reflected by the breasts 100a, while the rest pass through the breasts 100a (FIG. 3B) and reaches the plurality of transducers 1 of the transducer array 2 (FIG. 3C), and are received and converted into a reception signal (an electric signal) by the transducers 1. Further, a part of the ultrasonic waves 201 that reached the transducer array 2 are specularly reflected by a smooth surface of the transducer array 2 (FIG. 3C), become reverberation waves (reflection waves) 202, and reach the transducer array 2 again (FIG. 3E). The transducers 1 where the reverberation waves 202 reached converts the reverberation waves into a reception signal. Therefore, reception signals of the ultrasonic waves 201 and the reverberation waves 202 are output from the transducers 1 where the ultrasonic waves and the reverberation waves reached at respective reaching timings.

The receiver 32 receives the reception signal received by the transducers 1 selected by the transducer selector 4, amplifies the signal, and outputs the signal to the arithmetic unit 52. At this time, the transmission and reception separator 33 prevents the transmission signal output from the transmitter 31 from being input to the receiver 32 as the electric signal due to reflection or the like.

The arithmetic unit 52 generates an ultrasonic CT image by performing arithmetic processing on the reception signal output from the receiver 32, and displays the ultrasonic CT image on the display device 10.

The input and output unit 9 receives the imaging conditions and the like from the user.

In the present embodiment, since the number of transceivers 3 (for example, 256 ch) is smaller than the number of transducers 1 (for example, 2048 ch), the number of transducers 1a connected to the transmitter 31 by the transducer selector 4 is a predetermined number of 256 or less (here, 1 ch).

In addition, the number of transducers 1 connected to the receiver 32 by the transducer selector 4 for each reception is a predetermined number of 256 ch or less (here, 256 ch). In the present embodiment, as shown in FIG. 2, seven groups (hereinafter referred to as first to seventh reception modules) for every 256 adjacent channels are set in the transducers 1 (for example, 2048 ch) arranged in the transducer array 2 in a ring shape. The transducers 1a used for transmission are located in centers of the plurality of transducers 1 in which the first to seventh reception modules are not provided. Transmission is repeated seven times from one transmission transducer 1a, and the transducer selector 4 selects the transducers 1 in the first reception module to the seventh reception module in order and connects the transducers 1 to the receiver 32 for each reception. Accordingly, the receiver 32 receives and processes the reception signal obtained by receiving the ultrasonic waves from one transmission transducer 1a in the order of the first reception module to the seventh reception module for seven times. Accordingly, the transmission and reception (also called one view) from a position of one transmission transducer 1a is completed. This transmission and reception operation is repeated over the entire circumference of the ring-shaped transducer array 2 while sequentially changing positions of the transmission transducers 1a by a predetermined angle, and the number of reception signals required for reconstruction of the ultrasonic CT is acquired.

A combination of the transducers 1a used for transmission and the reception modules used for reception when a series of transmission and reception are repeated, and transmission and reception timings thereof are predetermined and stored in the storage unit 8.

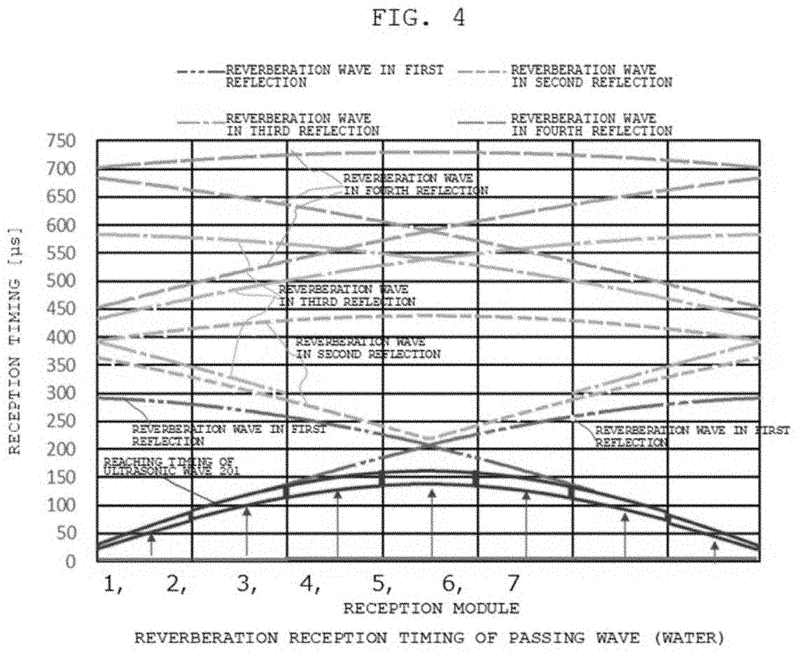

Here, a method for determining the combination of the transducers 1a and the reception modules and the transmission and reception timings thereof will be described with reference to FIGS. 4 to 7.

FIG. 4 is a graph showing timings at which wavefronts of the ultrasonic waves 201 transmitted from the transducers 1a and passed through a measurement space surrounded by the transducer array 2 reach the transducers 1 in the first to seventh reception modules (see FIGS. 3A to 3C), and timings at which wavefronts of the reverberation waves 202 reflected by a mirror surface of the transducer array 2 reach the transducers 1 in the first to seventh reception modules. The graph is obtained by the inventors by arithmetic assuming that the ultrasonic waves are specularly reflected in the transducer array 2 to generate the reverberation waves. It is assumed that the measurement space inside the transducer array is filled with water.

In FIG. 4, the transmission timing is 0 μs. A horizontal axis indicates the first to seventh reception modules, and a vertical axis indicates timings at which the ultrasonic waves 201 and the reverberation waves 202 reach the respective reception modules with elapsed time from the transmission time point. The vertical axis further indicate respective reception timings of the reverberation waves 202 reflected once to four times by the transducer array 2. The reaching timing of the ultrasonic waves 201 is indicated by double lines. This is because, when the breasts 100a are placed in the measurement space inside the transducer array, a sound speed of a tissue (such as fat, mammary gland) of the breasts 100a is different from a sound speed of water. Accordingly, the reaching timing is represented by a range of the reaching timing between a maximum sound speed and a minimum sound speed of the tissue. That is, when the receiver 32 receives the reception signal during the time between the double lines, the reception signal from the tissue of the breasts 100a can be acquired. A Reaching timing of the reverberation waves 202 also has a range of the reaching timing between the maximum sound speed and the minimum sound speed, and display of the range is omitted because the graph would become complicated otherwise, and the reaching timing at the sound speed of water is shown by a line.

As is clear from FIG. 4, after the ultrasonic waves 201 are transmitted, passing waves of the ultrasonic waves 201 first reach the reception modules, and then the reverberation waves 202 reach the reception modules. The reaching timing of the ultrasonic waves 201 is different from the reaching timing of the reverberation waves 202 depending on distances between the transmission transducers 1a and the reception modules.

Therefore, in the related art, after first transmission is performed, the ultrasonic waves 201 are received by the first reception module, and second transmission is performed after the reverberation waves 202 reach the first to seventh reception modules, thereby preventing the reverberation waves 202 by the first transmission from overlapping with the ultrasonic waves 201 by the second transmission and reaching the second reception module. Therefore, it is necessary to set the transmission interval to 800 μs or more.

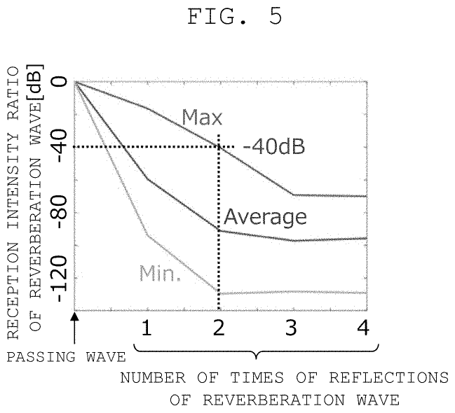

The inventors obtained a relationship between the number of times of reflection of the reverberation waves 202 and an intensity of the reception signal by arithmetic. Results are shown in the graph of FIG. 5. As is clear from FIG. 5, an average reception intensity of the reverberation waves 202 is greatly attenuated as the number of times of reflection increases as compared with the passing waves (ultrasonic waves 201). Since the reverberation waves 202 after second reflection is −40 dB as compared with the passing waves (ultrasonic waves 201) even at the maximum reception intensity, the reverberation waves 202 after the second reflection are ignored in the present embodiment.

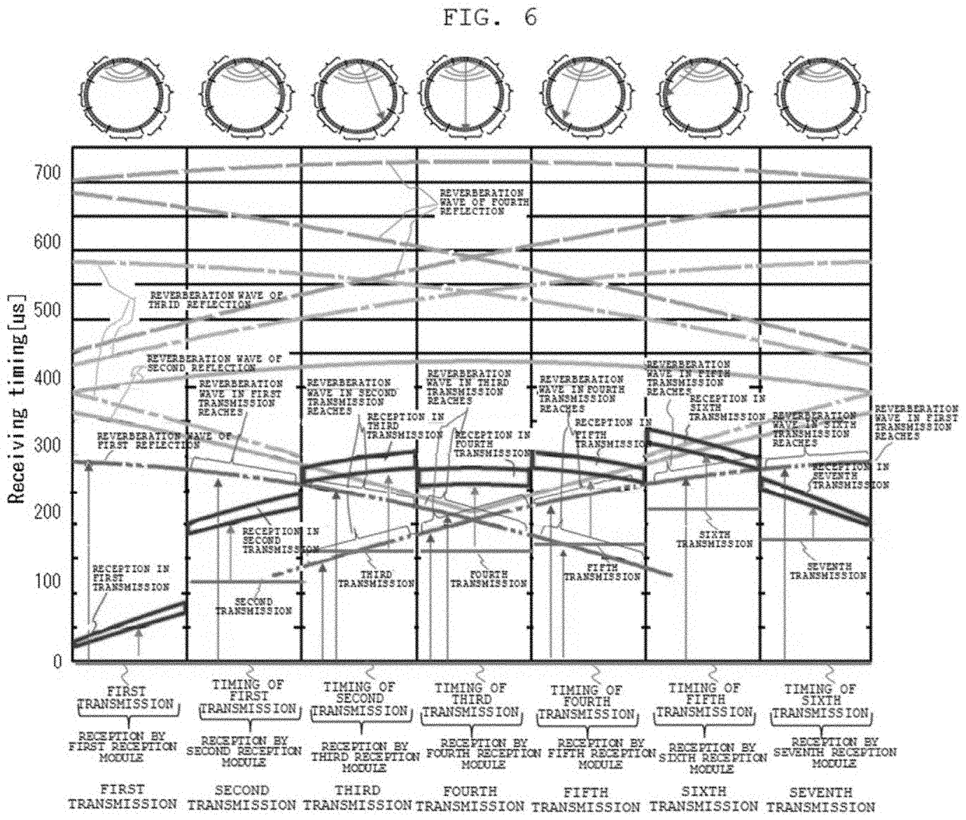

Therefore, in the present embodiment as shown in FIG. 6, the transmission timing of the currently transmitted ultrasonic waves is determined such that the ultrasonic waves in the current transmission reach a current reception module while avoiding the reaching timing of the reverberation waves 202 in the first reflection generated by the previous transmission.

For example, as shown in FIG. 6, the reception of the ultrasonic waves 201 transmitted at the second time by the second reception module is performed immediately before the reverberation waves 202 of the ultrasonic waves 201 transmitted at the first time reach the second reception module. Therefore, the timing of the second transmission is determined based on transmission and reception conditions such that the ultrasonic waves 201 in the second transmission reach the second reception module immediately before the reverberation waves 202 reach the second reception module.

The transmission and reception conditions here determine the timing of the second transmission in consideration of a distance between the transmission transducers 1a and the second reception module and a sound speed of the ultrasonic waves 201 that are based on the positions of the transducers 1a used for the second transmission and the position of the second reception module used for the second reception. In addition, regarding a sound speed of the ultrasonic waves 201, it is desirable to consider the maximum sound speed and the minimum sound speed predetermined for components of the breasts 100a. It is more desirable to consider a wavefront shape, a waveform, a signal length, etc. of the transmitted ultrasonic waves as the transmission and reception conditions.

In addition, the reception of the ultrasonic waves 201 transmitted at the third time by the third reception module is performed immediately after the reverberation waves 202 of the ultrasonic waves 201 transmitted at the second time reach the third reception module. Therefore, a timing of the third transmission is determined in consideration of a distance between the transmission transducers 1a and the third reception module and the sound speed of the ultrasonic waves 201 such that the ultrasonic waves 201 in the third transmission reach the third reception module immediately after the reverberation waves 202 in the second transmission reach the third reception module.

Similarly, the reception of ultrasonic waves 201 transmitted at the fourth, fifth and sixth times by the fourth, fifth and sixth reception modules is performed immediately after the reverberation waves 202 of the ultrasonic waves 201 transmitted at the third, fourth and fifth times reach the fourth, fifth and sixth reception modules, respectively. Therefore, timings of the fourth, fifth, and sixth transmissions are determined in consideration of distances between the transmission transducers 1a and the fourth, fifth, and sixth reception modules and the sound speed of the ultrasonic waves 201 such that the ultrasonic waves 201 in the fourth, fifth, and sixth transmission reach the fourth, fifth, and sixth reception modules immediately after the reverberation waves 202 transmitted at the third, fourth and fifth times reach the fourth, fifth and sixth reception modules.

The reception of the ultrasonic waves 201 transmitted at the seventh time by the seventh reception module is performed immediately before the reverberation waves 202 of the ultrasonic waves 201 transmitted at the sixth time reach the seventh reception module. Therefore, a timing of the seventh transmission is determined in consideration of a distance between the transmission transducers 1a and the seventh reception module and the sound speed of the ultrasonic waves 201 such that the ultrasonic waves 201 in the seventh transmission reach the seventh reception module immediately before the reverberation waves 202 reach the seventh reception module.

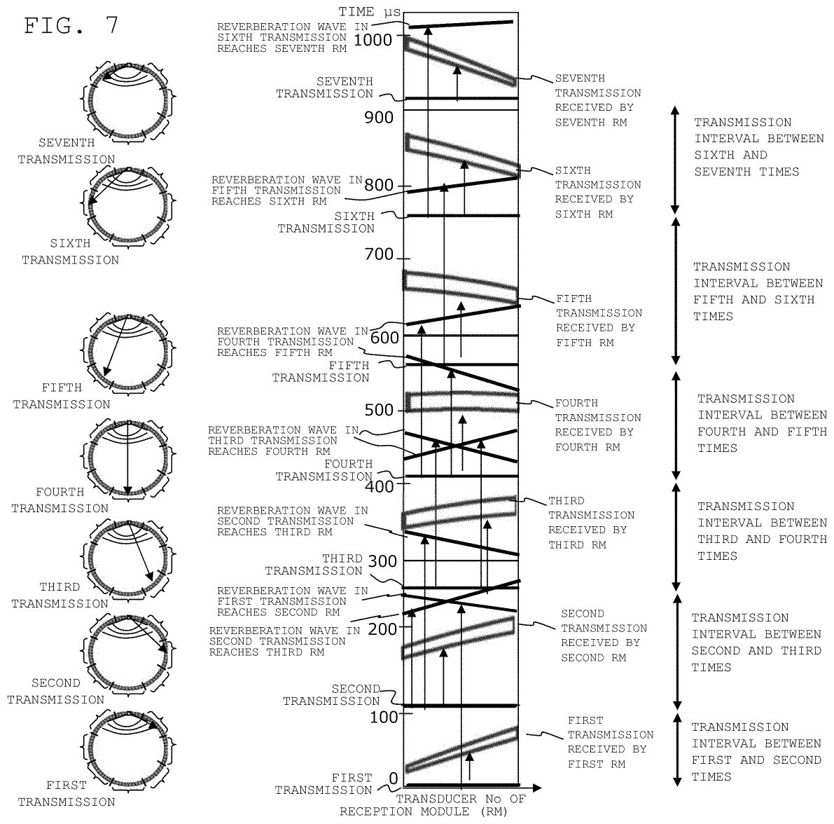

FIG. 7 shows time-series representations of the seven transmission timings and reception timings determined as shown in FIG. 6. As is clear from FIG. 7, in the present embodiment, it is determined that current ultrasonic waves are received immediately before or immediately after the previous reverberation waves 202 reach the current reception module while avoiding a timing at which the reverberation waves 202 reach the current reception module. Therefore, the transmission interval is different for each transmission. In FIG. 7, the reception modules are abbreviated as RM.

In addition, instead of performing the next transmission after a series of reverberation waves 202 reach as in the related art, the next transmission and reception are performed before the previous reverberation waves 202 reach (second transmission, seventh transmission), or the next transmission is performed before the previous reverberation waves 202 reach, and the ultrasonic waves 201 are received immediately after the reverberation waves 202 reach (third to sixth transmissions).

Therefore, the transmission interval of the present embodiment is set to about 110 to 220 μs, which is significantly shorter than the 800 μs in the related art. Therefore, seven transmissions can be performed within 1 ms as shown in FIG. 6, and imaging time can be significantly shortened.

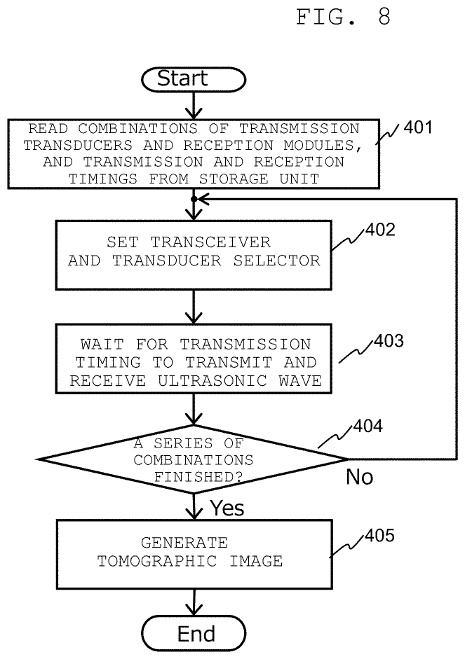

Next, operation of each unit when imaging the breasts 100a using the ultrasonic CT device for breasts of the present embodiment will be described with reference to a flowchart of FIG. 8. Here, as an example, a passing wave image of the breasts 100a is imaged.

The combinations of the transducers 1a used for transmission and the reception modules used for reception when the series of transmission and reception are repeated, and the transmission and reception timings thereof determined as shown in FIG. 7 are stored in the storage unit 8 in a form of a table or the like.

FIG. 7 shows only a timing at which transmission from the transmission transducers 1a at one position to the first to seventh reception modules is repeated seven times and received by respective reception modules. To generate a cross-sectional CT image, it is necessary to transmit and receive ultrasonic waves from an entire circumferential direction while shifting the transmission transducers 1a and the first to seventh reception modules by a predetermined angle (while changing a view angle). The storage unit 8 stores the combinations of the transducers 1a and the reception modules used for reception when transmission and reception are repeated in all the views, and the transmission and reception timings thereof as a series of combinations.

The signal processing unit 5 is a computer or the like including a processor such as a central processing unit (CPU) or a graphics processing unit (GPU), and a memory. The CPU reads and executes a program stored in the memory, so that functions of units of the signal processing unit 5 can be implemented by software, and a part or all of the functions can be implemented by hardware. For example, the signal processing unit 5 is a custom IC such as an application specific integrated circuit (ASIC) or a programmable IC such as a field-programmable gate array (FPGA), so that a circuit may be designed to implement the functions of the units of the signal processing unit 5.

The subject 100 is placed on bed 101 facing downward and the breasts 100a of the subject 100 are inserted into the container 103.

### Step 401

When a user inputs an instruction to start imaging to the input and output unit 9, the control unit 5 reads from the storage unit 8 a series of combinations of the transmission transducers 1a and the reception modules used for reception, and the transmission and reception timings (reception period) thereof (Step 401).

### Step 402

The control unit 5 sets a combination of a first transmission transducer 1a and reception modules used for reception in the series of combinations in the transducer selector 4. Accordingly, the transducer selector 4 connects the transducer 1a to the transmitter 31 and connects the set reception modules to the receiver 32 (Step 402).

### Step 403

Next, the control unit 51 waits for the transmission timing read from the storage unit 8, and outputs an electric signal to the transmitter 31 at the transmission timing. Accordingly, the transmitter 31 outputs the transmission signal to the transducer 1a via the transducer selector 4, and the transducer 1a transmits the ultrasonic waves 201.

The ultrasonic waves 201 pass through the breasts 100a and reach the first reception module, and is converted into a reception signal by the transducer 1 in the first reception module. Under the control of the control unit 51, the transducer selector 4 passes the reception signal of the transducer 1 in the first reception module at the reception timing (reception period) read in step 401 to the receiver 32 (Step 403).

Accordingly, the receiver 32 receives the reception signal output from the transducer 1 in the first reception module, amplifies the signal, and outputs the amplified signal to the arithmetic unit 52. The arithmetic unit 52 stores the signal in the storage unit 8.

### Step 404

Next, the control unit 51 determines whether the combination set in step 402 is a last combination of the transmission transducer 1a and the reception module used for reception read from the storage unit 8 (Step 404). In a case of the first transmission, since the combination is not the last, the process returns to step 402, the combination of the transmission transducer 1a of the second transmission and the second reception module used for reception is set in the transducer selector 4, and step 403 is performed.

Accordingly, as shown in FIGS. 6 and 7, before the reverberation waves 202 in the first transmission reach the second reception module, the second transmission from the transducer 1a and the reception by the second reception module are performed.

Returning to step 402 again, the combination of the transmission transducer 1a and the third reception module used for reception, and the transmission and reception timings (reception period) thereof are set in the transducer selector 4, and step 403 is performed.

Accordingly, as shown in FIGS. 6 and 7, before the reverberation waves 202 in the second transmission reach the third reception module, the third transmission from the transducer 1a is performed, and the third reception module receives the ultrasonic waves of the third transmission immediately after the reverberation waves 202 reach the third reception module.

Similarly, steps 402 to 403 are repeated, transmission and reception are performed for all the series of combinations stored in the storage unit 8. When the reception by the first to seventh reception modules is completed, the process proceeds to step 405.

### Step 405

The arithmetic unit 52 performs processing on reception signals obtained in each view, thereby generating a cross-sectional image (passing wave image) of the breasts 100a by a known method (Step 405).

In addition, by repeating the above steps 401 to 404 while changing a position of the transducer array 2 at a predetermined pitch in a depth direction, a tomographic image of the breasts 100a can be generated at all predetermined depths, and three-dimensional data of the breasts 100a can be acquired.

As described above, according to the present embodiment, since the transmission timing is set for each transmission so that the timing at which the ultrasonic waves reach the reception transducers avoids the timing at which the previous reverberation waves reach the reception transducers, the transmission can be performed without waiting until the reverberation waves are sufficiently attenuated, and the imaging time can be shortened. Accordingly, a throughput of inspection can be increased.

### Second Embodiment

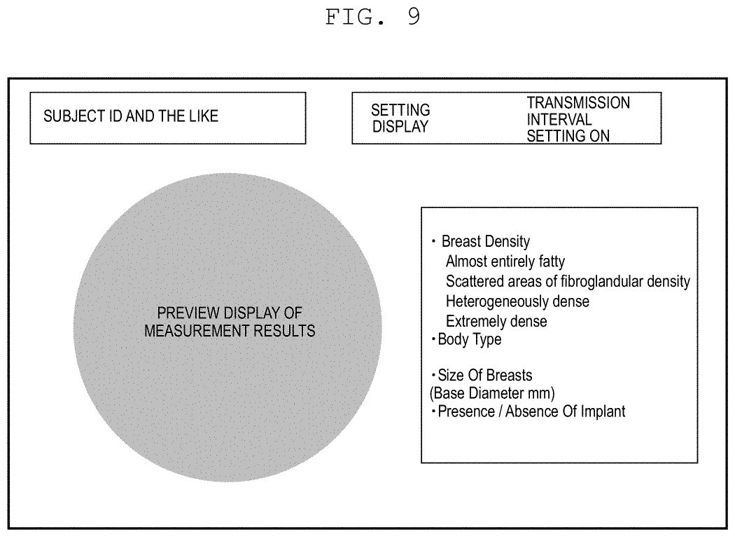

An ultrasonic CT device for breasts according to the second embodiment will be described with reference to FIG. 9.

In the first embodiment, as shown in FIGS. 6 and 7, a configuration is described in which the transmission and reception timings are set in advance based on the arithmetic such that the ultrasonic waves 201 reach the reception modules while avoiding the timing at which the reverberation waves 202 reach the reception modules, and in which the transmission and reception timings are stored in the storage unit 8. In the first embodiment, the timing at which the ultrasonic waves 201 reach the reception modules and the timing at which the reverberation waves 202 reach the reception modules differ depending on the mammary gland density, a size of the breasts 100a, a body type of the subject 100, and presence or absence of an implant.

Therefore, in the second embodiment, for each of a plurality of types of mammary gland densities, the size of the breasts 100a of the subject 100, and the body type of the subject 100, and for each combination thereof, a series of transducers and reception modules and the transmission and reception timings thereof are set in advance based on the arithmetic and are stored in the storage unit 8.

Before step 401 in FIG. 4, the control unit 51 displays, for example, an input screen (GUI) as shown in FIG. 9 on the display device 10, and receives any selection or numerical value input of mammary gland densities (fatty, scattered mammary glands, unevenly high concentration, fairly high concentration) and input of the size of the breasts 100a, the body type of the subject 100, the presence or absence of the implant, and the like via the input and output unit 9.

Then, in step 401 of FIG. 4, when reading out the combination of the transmission transducers and the reception modules, and transmission and reception timings, the control unit 51 reads out a combination corresponding to the input mammary gland densities and the like.

Accordingly, even when the mammary gland densities, the size of the breasts 100a, and the body type of the subject 100 differ depending on the subject 100, an optimum combination of the transmission transducers and the reception modules, and the transmission and reception timings thereof can be set. Therefore, the imaging time can be shortened, and the reverberation waves 202 can be prevented from being received by the receiver 32, so that a reconstructed image can be generated with high accuracy.

A setting input of the mammary gland densities, the size of the breasts 100a, and the body type of the subject 100 is not limited to a method by manually input by the subject or an operator on a screen shown in FIG. 9, and the setting input also can be automatically input from a medical record or from another inspection device. For example, the setting input can automatically receive the mammary gland densities from a mammography device.

Configurations and operations of the ultrasonic CT device of the second embodiment other than those described above are the same as the configurations and operations of the first embodiment, and thus description thereof is omitted.

### Third Embodiment

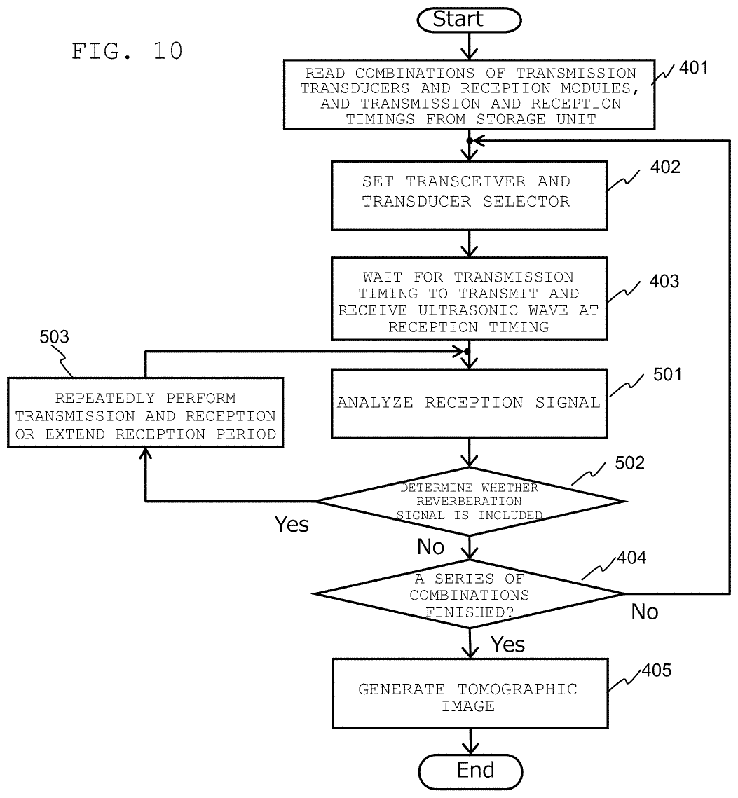

An ultrasonic CT device for breasts according to the third embodiment will be described with reference to FIG. 10.

As shown in FIG. 10, an imaging operation of the ultrasonic CT device of the present embodiment is the same as the flowchart of FIG. 8 of the first embodiment, and steps 501, 502, and 503 are added to a flowchart of FIG. 10.

In the third embodiment, in steps 501, 502, and 503, the control unit 51 analyzes reception signals during a predetermined reception period in real time to determine whether a reception signal of the reverberation waves 202 is included in the reception signals during the predetermined reception period. When the reception signal of the reverberation waves 202 is included, the control unit 51 changes at least one of the transmission and reception timings and repeatedly performs current transmission and reception. Alternatively, when the ultrasonic waves 201 are not arrived yet and will arrive from now on, only the reception is repeatedly performed. Accordingly, even when the reaching timing of the reverberation waves 202 used to determine the timings of the transmission and the reception of the reception modules stored in the storage unit 8 in advance deviates from an actual reaching timing of the reverberation waves 202, the reception signals not including the reception signal of the reverberation waves 202 are acquired. A specific description will be given below.

### Steps 401 to 403

As in the first embodiment, by performing steps 401 to 403, the receiver 32 receives the reception signal at the reception timing (reception period) of the first reception module from the transmission transducer 1a.

### Steps 501 and 502

The control unit 51 analyzes the reception signal acquired by the receiver 32 (Step 501), and determines whether the reception signal includes a peak of the reverberation waves 202 (Step 502). For example, when time-series reception signals include two peaks larger than a predetermined value, or when a time relationship (for example, continuous time relationship) of peaks of the reception signal of the adjacent transducers 1 includes not only a relationship assumed by the ultrasonic waves 201 but also a relationship assumed by the reverberation waves 202, the process proceeds to step 503 since it can be determined whether both the ultrasonic waves (passing waves) 201 and the reverberation waves 202 are received or only the reverberation waves 202 are received.

### Step 503

In step 503, the control unit 51 changes the transmission and reception timings and repeatedly performs current transmission and reception, or repeatedly performs only the reception.

For example, in step 502, when the control unit 51 determines that both the ultrasonic waves (passing waves) 201 and the reverberation waves 202 are received, the control unit 51 repeatedly performs arithmetic processing on the transmission and reception timings based on the time relationship between the reverberation waves 202 and the reception period, and changes the transmission and reception timings so that the reverberation waves 202 determined to be included in the reception signal can be avoided. Alternatively, as in the second embodiment, a plurality of combinations of a series of transducers and reception modules, and the transmission and reception timings thereof are stored in the storage unit 8, and the transmission and reception timings are reset by selecting the transducers from the combinations. The current transmission and reception will be repeatedly performed according to the changed transmission and reception timings.

In addition, for example, in the above step 502, when the control unit 51 determines that only the reverberation waves 202 are received and the ultrasonic waves (passing waves) 201 are not received, the control unit 51 extends the reception period and receives the ultrasonic waves 201 that will arrive from now on. That is, since the reception signals can be analyzed and determined in real time in steps 501 and 502 at the same time as step 403, the ultrasonic waves 201 originally to be received may not reach the reception modules even if it is determined that the reception signals include the reverberation waves 202. In this case, the control unit 51 may continue the reception by extending the reception period to a predetermined reception period in step 503, and a reception signal of the ultrasonic waves 201 that do not include the reverberation waves 202 in step 403 may be used for the arithmetic in step 405. When it is determined that the reverberation waves 202 and the ultrasonic waves 201 are mixed as a result of continuing the reception, the process proceeds to step 503, and the current transmission and reception are repeated.

In step 502, when the number of channels receiving the reverberation waves 202 is less than a predetermined threshold value, or when a peak intensity of the received reverberation waves 202 is sufficiently smaller than a peak intensity of the received ultrasonic waves 201, the control unit 51 can determine that a “degree of separation” of the ultrasonic waves 201 with respect to the reverberation waves 202 is higher than a predetermined value. In that case, the control unit 51 may proceed to step 404 without proceeding to step 503 and repeatedly performing the transmission and reception.

Specifically, when the reception modules have 256 channels and the number of channels receiving reception signals including the reverberation waves 202 is sufficiently small (for example, less than 10 channels), influence of the reverberation waves 202 on the tomographic image is minor since the reception signals obtained in all the view channels are processed to generate the tomographic image in step 405. Therefore, a threshold value of the number of channels as to whether to repeatedly perform the current transmission and reception is set in advance. When the number of channels receiving the reverberation waves 202 is less than the threshold value, the control unit 51 determines that the “degree of separation” is high, and proceeds to step 404 immediately. In addition, determination criteria of the degree of separation may include a threshold value of a ratio of a peak intensity of the reception signal determined to be the reverberation waves 202 to a peak intensity of the reception signal determined to be the ultrasonic waves 201. When a ratio of an intensity of the reverberation waves 202 to an intensity of the ultrasonic waves 201 is less than a threshold value, the control unit 51 determines that the degree of separation is high and proceeds to step 404 immediately. The control unit 51 may determine whether to repeatedly perform the transmission and reception by combining determination of the degree of separation based on the number of channels and determination of the degree of separation based on the peak intensity of the reverberation waves.

In step 503 described above, a configuration in which the control unit 51 sets the transmission and reception timings to be repeatedly performed has been described. Alternatively, the control unit 51 may display on a connected display device a display prompting a user to change at least one of a transmission timing and a reception timing, receive the changed timing desired by the user and perform the transmission and reception at that timing.

In the present embodiment, in steps 501 and 502, the control unit 51 analyzes the reception signal and determines whether the reverberation waves 202 are included in the reception signal received during the reception period. Alternatively, the control unit 51 may determine whether image quality is high by using a preview image (see FIG. 9) generated by the arithmetic unit 52. When the image quality is not high, it is determined that the image is affected by the reverberation waves 202, and the control unit 51 changes the transmission and reception timings and repeatedly performs transmission and reception of the cross section.

When the control unit 51 determines that the image quality is not high and the image is affected by the reverberation waves 202, the control unit 51 may display on the connected display device a display prompting the user to re-image the cross section by changing the transmission and reception timings, receive the changed transmission and reception timings desired by the user and perform transmission and reception at that timing. Alternatively, the control unit 51 may not make a determination, and may leave the determination to the user and receive that the cross section specified by the user is re-imaged at the transmission and reception timings desired by the user.

Configurations and operations of the ultrasonic CT device of the third embodiment other than those described above are the same as the configurations and operations of the first embodiment, and thus description thereof is omitted.

In the ultrasonic CT device of the third embodiment, the reception signal of the reverberation waves can be prevented from being mixed into the reception signal of the passing waves, and an ultrasonic CT image with a high resolution can be acquired in a short time.

### Fourth Embodiment

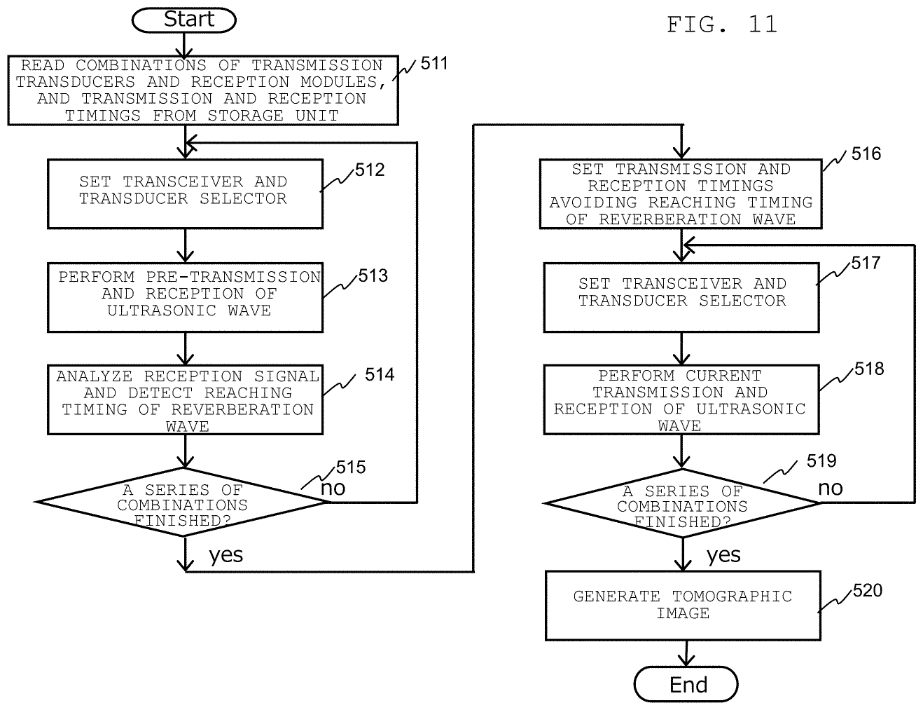

An ultrasonic CT device for breasts according to the fourth embodiment will be described with reference to FIG. 11.

The ultrasonic CT device of the present embodiment has the same configurations as the device of the first embodiment. Further, as shown in a flowchart of FIG. 11, the control unit performs predetermined pre-transmission and reception, obtains a timing at which the reverberation waves 202 reach the transducers used for reception (Steps 511 to 515), determines transmission and reception timings only this time or a series of transmission and reception timings based on the obtained reaching timing of the reverberation waves (Step 516), and performs current transmission and reception using the determined transmission and reception timings (Steps 517 to 520).

An example of control operations of the control unit 51 will be described with reference to FIG. 11.

### Steps 511 to 515

In the present embodiment, as steps 511 to 515 of the pre-transmission and reception, when a subject is placed, the control unit 51 performs a series of transmission and reception and analysis of reception signals in the same manner as steps 401 to 403 and 501 to 502 of the second embodiment, and detects reaching timings of the reverberation waves 202 for each transmission and reception.

In step 513, the control unit 51 may perform the pre-transmission and reception by extending the reception period of the reception timing shown in the second embodiment. Accordingly, even when the reverberation waves reach the transducers and are deviated from an assumed state, the reverberation waves 202 can be reliably received within the reception period, and the reaching timing of the reverberation waves 202 can be detected.

In addition, in step 514 of the pre-transmission and reception, the control unit 51 may detect not only the reaching timing of the reverberation waves 202 but also the reaching timing of the ultrasonic waves 201 by the same method.

In addition, in step 514, the control unit 51 may analyze the intensity of the reverberation waves 202, determine that the reverberation waves 202 hardly affect the image when the intensity is smaller than a predetermined threshold value, and ignore the reaching of the reverberation waves 202.

### Step 516

In step 516, the control unit 51 sets the reception timing to avoid the reaching timing of the reverberation waves 202 based on the reaching timing of the reverberation waves 202 obtained for each transmission and reception detected in step 514, and determines the transmission timing in consideration of positions of the transmission transducers 1a and the reception modules so that the ultrasonic waves 201 reach the reception module at the reception timing.

In step 514, when both reaching timings of the reverberation waves 202 and the ultrasonic waves 201 are detected, the control unit 51 can set the reception timing such that the reaching timing of the ultrasonic waves 201 is included in the reception timing while avoiding the reaching timing of the reverberation waves 202.

As in the second embodiment, a plurality of parameter sets of the transmission and reception timings may be prepared in advance, and the control unit 51 may select and set an optimum set from results of the pre-transmission and reception.

### Steps 517 to 520

In steps 517 to 520 of the current transmission and reception, the transmission and reception are performed in the same manner as in steps 402 to 405 of FIG. 8 of the first embodiment by using the transmission and reception timings set in step 516 to generate a tomographic image.

In steps 511 to 512 described above, a case is described in which the pre-transmission and reception and the current transmission and reception are both performed according to combinations of a predetermined series of transducers and the reception modules as in the first embodiment. Alternatively, the invention is not limited to this configuration, and the pre-transmission and reception may perform transmission and reception operations different from that of the current transmission and reception. For example, by transmitting the ultrasonic waves 201 from the position of one transducer 1a, measuring reaching timings of the ultrasonic waves 201 and the reverberation waves 202 reaching each reception module, a graph as shown in FIG. 4 may be obtained, and based on this graph, the transmission and reception timings of each transmission may be determined to avoid the reverberation waves reflected at least once in step 516.

Configurations and operations of the ultrasonic CT device of the fourth embodiment other than those described above are the same as the configurations and operations of the first and third embodiments, and thus description thereof is omitted.

In the ultrasonic CT device of the fourth embodiment, since the reverberation waves can be actually measured by the pre-transmission and reception to determine the transmission and reception timings of each transmission, in the current transmission and reception, the reception signal of the reverberation waves can be prevented from being mixed into the reception signal of the passing waves, and an ultrasonic CT image with a high resolution can be acquired in a short time.

In addition, since the reaching timing of the ultrasonic waves 201 can be measured by the pre-transmission and reception and the transmission and reception timings can be set, the reverberation waves 202 can be avoided, reception accuracy of the ultrasonic waves 201 can be improved, and an ultrasonic CT image with higher resolution can be acquired.

### MODIFICATIONS

In the first to fourth embodiments described above, when the passing waves are received and the passing wave image is imaged, the passing waves are received at a timing avoiding the reverberation waves. Alternatively, it is also possible to avoid the reverberation waves and receive the reflection waves when a reflection wave image is to be imaged.

What is claimed is:

1. An ultrasonic CT device comprising:
a transducer array in which a plurality of transducers are arranged to surround a region where a measurement object is placed;
a transmitter configured to output a transmission signal to the transducers to transmit ultrasonic waves;
a receiver configured to receive and process reception signals obtained by a predetermined number of transducers of the transducer array which receive the ultrasonic waves from the region that received the ultrasonic waves, and output; and
a control unit configured to set combinations of the transducers from which the ultrasonic waves are transmitted in the transmitter and the transducers that receive the reception signals in the receiver sequentially according to a predetermined series of combinations to control repetition of transmission and reception of the ultrasonic waves, wherein
when the transmission and reception are repeated, the control unit controls a timing of current transmission such that a timing at which reverberation waves, which are ultrasonic waves transmitted in previous transmission and are reflected by the transducer array at least once, reach transducers used for current reception deviates from a timing at which ultrasonic waves transmitted in the current transmission reach the transducers used for the current reception.

2. The ultrasonic CT device according to claim 1, wherein
in a series of repetition of the transmission and reception, a timing of n-th transmission is predetermined based on transmission and reception conditions including positions of transducers used for (n−1)-th transmission, a timing of (n−1)-th transmission, positions of transducers used for the n-th transmission and positions of transducers used for n-th reception.

3. The ultrasonic CT device according to claim 2, wherein
the transmission and reception conditions include a maximum sound speed and a minimum sound speed predetermined for components of the measurement object.

4. The ultrasonic CT device according to claim 1, wherein
the receiver processes reception signals of passing waves of the ultrasonic waves passing through the region where the measurement object is placed,
the control unit controls the timing of the current transmission such that a timing at which the passing waves of currently transmitted ultrasonic waves reach the transducers used for the current reception deviates from a timing at which reverberation waves of passing waves of previously transmitted ultrasonic waves reach the transducers used for the current reception.

5. The ultrasonic CT device according to claim 1, wherein
the control unit further sets a reception period of the reception signals by the receiver for each combination of transducers used for transmission and transducers used for reception.

6. The ultrasonic CT device according to claim 1, wherein
the control unit analyzes the reception signals during a predetermined reception period in real time to determine whether a reception signal of the reverberation waves is included in the reception signals during the predetermined reception period, and repeatedly performs the current transmission and reception when the reception signal of the reverberation waves is included.

7. The ultrasonic CT device according to claim 1, wherein
the control unit analyzes the reception signals during a predetermined reception period in real time to determine whether a reception signal of the reverberation waves is included in the reception signals during the predetermined reception period, and acquires a reception signal of the ultrasonic waves by extending the reception period and continuing the current reception when the reception signal of the reverberation waves is included and the reception signal of the ultrasonic waves is not included.

8. The ultrasonic CT device according to claim 6, wherein
the control unit determines, when the reception signal of the reverberation waves is included in the reception signals during the reception period, a degree of separation between the reverberation waves and the ultrasonic waves, and determines whether to repeatedly perform the transmission and reception based on the degree of separation.

9. The ultrasonic CT device according to claim 6, wherein
the control unit determines, when the number of transducers that received the reverberation waves is less than a predetermined threshold value, and/or when a ratio of a peak intensity of the reception signal of the reverberation waves to a peak intensity of a reception signal of the ultrasonic waves is less than a predetermined threshold value, that a degree of separation is higher than a predetermined value and determines not to repeatedly perform the transmission and reception.

10. The ultrasonic CT device according to claim 1, wherein
the control unit analyzes the reception signals during a predetermined reception period in real time to determine whether a reception signal of the reverberation waves is included in the reception signals during the predetermined reception period, and when the reception signal of the reverberation waves is included, the control unit displays on a connected display device a display which prompts a user to change at least one of a transmission timing and a reception timing.

11. The ultrasonic CT device according to claim 1, further comprising:
an arithmetic unit configured to generate an image of the measurement object with the reception signals obtained by the receiver by repeatedly performing the transmission and reception, wherein
the control unit analyzes the image to determine whether the reverberation waves also affect the image, when the reverberation waves affect the image, the control unit changes the transmission timing and repeatedly performs the transmission and reception.

12. The ultrasonic CT device according to claim 1, further comprising:
an arithmetic unit configured to generate an image of the measurement object with the reception signals obtained by the receiver by repeatedly performing the transmission and reception, wherein
the control unit determines whether the reverberation waves affect the image, when the reverberation waves affect the image, the control unit displays on a connected display device the image and a display which prompts a user to change the transmission timing when the reverberation waves affect the image.

13. The ultrasonic CT device according to claim 1, wherein
the control unit performs predetermined pre-transmission and reception and obtains a timing at which the reverberation waves reach the transducers used for reception, and the control unit determines a predetermined transmission timing or a series of transmission timings based on the obtained reaching timing of the reverberation waves, and the control unit performs current transmission and reception at the determined transmission timing.

14. The ultrasonic CT device according to claim 13, wherein
the control unit further obtains a timing at which the ultrasonic waves reach the transducers used for reception by the pre-transmission and reception, and the control unit sets timings of the current transmission and reception such that a reception timing coincides with the reaching timing of the ultrasonic waves avoiding the reaching timing of the reverberation waves.

15. The ultrasonic CT device according to claim 13, wherein
the control unit performs both the pre-transmission and reception and the current transmission and reception according to the predetermined series of combinations.

16. The ultrasonic CT device according to claim 1, wherein
the control unit controls the timing of the current transmission based on at least one of a type of body shape, presence or absence of an implant, and a mammary gland density, of the measurement object that are input in advance.

17. A method for controlling an ultrasonic CT device including a transducer array in which a plurality of transducers are arranged to surround a region where a measurement object is placed, the method comprising:
repeatedly performing a process of sequentially selecting combinations of transducers used for transmission of ultrasonic waves and transducers used for reception of the ultrasonic waves according to a predetermined series of combinations, transmitting the ultrasonic waves from the selected transducers, and receiving the ultrasonic waves; and
controlling a timing of current transmission such that a timing at which reverberation waves, which are ultrasonic waves transmitted in previous transmission and are reflected by the transducer array at least once, reach transducers used for current reception deviates from a timing at which ultrasonic waves transmitted in the current transmission reach the transducers used for the current reception.

18. A program for controlling an ultrasonic CT device including a transducer array in which a plurality of transducers are arranged to surround a region where a measurement object is placed, wherein
the program causes a computer to:
repeatedly perform a first step of selecting combinations of transducers used for transmission of ultrasonic waves and transducers used for reception of the ultrasonic waves according to a predetermined series of combinations, and a second step of transmitting the ultrasonic waves from the selected transducers and receiving the ultrasonic waves; and
control, in an n-th first step and second step, a transmission timing in the n-th second step such that a timing at which reverberation waves, which are ultrasonic waves transmitted in the second step in an n−1th time and earlier and are reflected by the transducer array at least once, reach transducers used for n-th reception, deviates from a timing at which ultrasonic waves transmitted in the n-th second step reach the transducers used for the reception of the n-th second step.
